(19) United States
(12) Patent Application Publication
(10) Pub. No.: US 2025/0339071 A1
(43) Pub. Date: Nov. 6, 2025

(54) METHOD FOR IDENTIFYING CLINICAL ISSUES IN MENTAL HEALTH USING PHYSICAL BUCKETS AND TOKENS

(71) Applicant: BRANDON BROWN, CINCINNATI, OH (US)

(72) Inventor: BRANDON BROWN, CINCINNATI, OH (US)

(21) Appl. No.: 18/655,913

(22) Filed: May 6, 2024

(51) Int. Cl. A61B 5/16; A61B 5/00

(52) U.S. Cl. CPC A61B 5/165; A61B 5/742

(57) ABSTRACT

The inventive embodiments relate to clinical therapy methods, particularly those involving the identification and management of a patient's negative emotions or mindsets during a therapy session. Specifically, this invention pertains to the field of mental health diagnostics and therapy. More specifically, it relates to a method for identifying and managing mental health issues using a system of physical buckets and tangible tokens.

### FIELD OF THE INVENTION

The present invention relates to clinical therapy methods, particularly those involving the identification and management of a patient's negative emotions or mindsets during a therapy session. Specifically, this invention pertains to the field of mental health diagnostics and therapy. More specifically, it relates to a method for identifying and managing mental health issues using a system of physical buckets and tangible tokens.

### BACKGROUND OF THE INVENTION

Clinical therapy often involves addressing a patient's negative emotions or mindsets. However, traditional methods may lack the ability to quantify or track these emotional states, making it difficult to assess the effectiveness of the therapy over time. There is a need for a method that can facilitate the tangible tracking of a patient's negative emotional states to improve the management and adaptation of clinical therapy plans.

### SUMMARY OF THE INVENTION

The present invention provides a method for facilitating the identification and management of a patient's negative emotions or mindsets during a clinical therapy session. The method includes engaging the patient in dialogue, utilizing physical buckets labeled with the identified emotions, discussing tangible objects placed in the buckets representing specific incidents or feelings, tracking the intensity and prevalence of these emotions, and adapting the therapy plan based on the tracked progress.

In another embodiment of the present invention, invention introduces a novel method for identifying one or more clinical issues in a patient's mental health by using a plurality of physical buckets and tangible items or tokens. This method facilitates a tactile and visual approach to mental health diagnosis and therapy planning.

### BRIEF DESCRIPTION OF THE FIGURES

The various exemplary embodiments of the present invention, which will become more apparent as the description proceeds, are described in the following detailed description in conjunction with the accompanying drawings, in which:

FIG. 1 is a perspective view of multiple labeled buckets with objects therein exemplifying their significance for a clinical

### DETAILED DESCRIPTION OF THE INVENTION

The following description is provided as an enabling teaching of the present systems, and/or methods in its best, currently known aspect. To this end, those skilled in the relevant art will recognize and appreciate that many changes can be made to the various aspects of the present systems, and/or methods described herein, while still obtaining the beneficial results of the present disclosure. It will also be apparent that some of the desired benefits of the present disclosure can be obtained by selecting some of the features of the present disclosure without utilizing other features.

Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

As used throughout, the singular forms “a”, “an” and “the” include plural referents unless the context clearly dictates otherwise. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word “or” as used herein means any one member of a particular list and also includes any combination of members of that list. Further, one should note that conditional language, such as, among others, “can”, “could”, “might”, or “may”, unless specifically stated otherwise, or otherwise understood within the context as used, is generally intended to convey that certain aspects include, while other aspects do not include, certain features, elements and/or steps.

Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods.

Thus, if there are a variety of additional steps that can be performed it is understood that each of these additional steps can be performed with any specific aspect or combination of aspects of the disclosed methods.

The term “balls” herein refers to multi-shaped objects placeable into a bucket or other like container for use of the embodiments herein. The balls may indeed be ball shaped objects which are handheld (or holdable in one or two hands). Such “balls” may also be of any conceivable shape for placement into a suitable container like a bucket or bin. Persons of skill in the art will readily recognize and understand that the shape of the balls can be any conceivable shape desired by a manufacturer, clinician or patient. Such shapes include but are not limited to spheres, triangles, polygonals, squares, and the like.

One embodiment of the method begins by engaging the patient in a dialogue to identify the patient's most significant negative emotions or mindsets. This discussion sets the foundation for the personalized therapy approach.

Following the dialogue, a plurality of actual, physical buckets is provided. Each bucket is to be labeled by the patient or therapist, correlating with one of the identified negative emotions or mindsets. Each labeled bucket is discussed between the therapist and the patient. The discussion includes the assignment of tangible objects into each bucket, with each object representing a specific incident, thought, or feeling associated with the corresponding negative emotion or mindset.

The method includes tracking the intensity and prevalence of the identified negative emotions or mindsets by analyzing the quantity and nature of objects assigned to each bucket during the therapy session. The outcomes of the analysis are utilized to guide the therapeutic conversation, aiming to address, mitigate, or understand the underlying causes of the negative emotions or mindsets identified.

Finally, the clinical therapy plan is adapted based on the progress observed through the tracking of object assignments to the buckets. This provides a tailored approach to the patient's mental health care.

In another embodiment herein, the method involves providing a plurality of actual, physical buckets, where each bucket represents a specific mental health issue such as anger, depression, guilt, anxiety, fear, bitterness, and the like. Patients are instructed to assign tangible items or tokens into each bucket based on personal experiences, feelings, or thoughts that correspond to the mental health issue represented by the bucket.

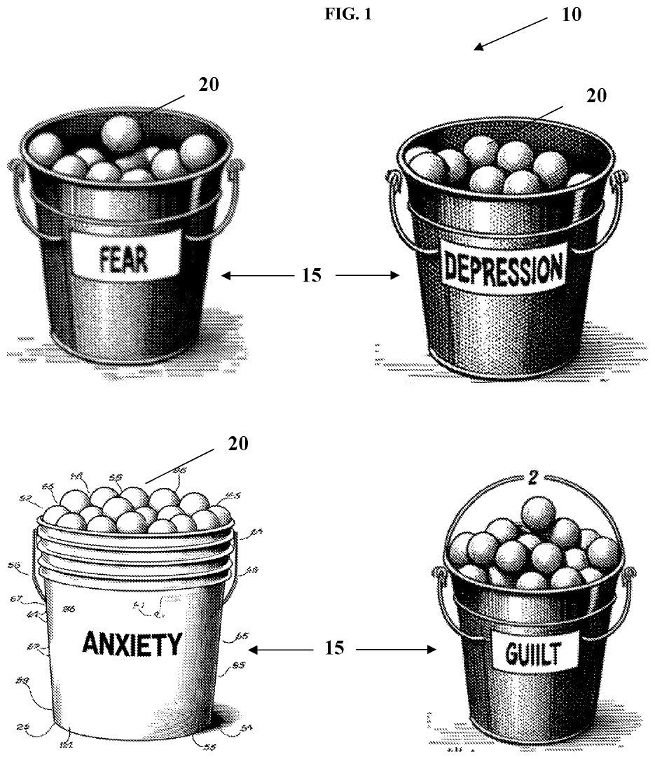

FIG. 1 provides an exemplary visual use of the method 10 herein. As shown, various buckets 15 are provided each labeled with a particular pathology, e.g., fear, depression, guilt and anxiety. The number of buckets used, and their labeling are at the discretion of both the practitioner (e.g., the psychiatrist/psychologist) and the patient.

In practice, a psychologist identifies the major issues with which a patient contends. Once completed, the patient can then assign weight to each issue using, for example, balls 20 placeable into each bucket as submitted therein by a patient. As the practitioner clinically examines the patient, the patient can place balls 20 into a bucket that aligns most with a discussed issue. By the end of the session, the practitioner (i.e., clinician) and patient will have a keen visual understanding not only of the major issues but also of the severity of each identified pathology.

It is also possible that by use of method 10 herein, a clinician is able to identify one or more issues not identified from an initial clinical query. In such revelation, a new issue can be added or used to replace an issue previously thought to be a profound part of the initial issue set. Also, once the buckets are filled, the total set is re-evaluated for importance and ranked. If there are lightly filed or unfilled buckets, those issues can be held in abeyance temporarily or permanently while the major identified issues are treated.

Herein, a healthcare professional analyzes the distribution of items or tokens among the buckets to identify predominant mental health issues, focusing on the quantity and nature of items placed in each bucket. The patient engages in a therapeutic dialogue focused on the outcomes of the distribution analysis to further explore and understand the patient's mental health issues. A tailored clinical therapy plan is developed based on the identified predominant mental health issues, incorporating strategies for addressing each identified issue.

This therapeutic tool (i.e., method) allows the client and therapist to narrow down the most problematic challenges that the client is currently experiencing. In order to address the client's most significant symptoms, the client will be able to use this tool as a means of focusing on the primary issue(s) identified in the session. The tool is comprised of individual buckets that can be labeled to reflect specific life matters that the client is facing at the time of the session. For example, the buckets could be labeled as family, friends, relationship, school, work, finances, health, children, parents, or household. During this step, the therapist will write down each the life matters and notate the details discussed.

Once all the necessary buckets are labeled, the remaining (unlabeled buckets) are removed and set aside. Then the client will begin to toss balls into each bucket while discussing the various challenges within their life (i.e. family matters, misunderstandings with friends, relationship dynamics, financial loss, social life, difficulties at school, issues with a teacher, problems at work, time management challenges, or co-parenting dynamics) that will be contained in the specific bucket. After the balls have been distributed the next step would be to identity which bucket has the most balls. The therapist would subsequently set aside all the other buckets and pull the primary bucket up to the center.

During this portion of the process, the therapist will proceed with discussing the individual issues associated with the balls in the primary bucket. This is helpful to reflect the information back to the client for clarity. The therapist will also begin the process of identifying details specific to the identified issues in addition to potential solutions for said issues, within the timeframe of the session. If it is possible, the therapist will then pull the secondary bucket containing the next significant number of balls and begin to discuss the details regarding that life matter. Should time permit, this process can be repeated and provide collaborative documentation opportunities for therapist and client.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for facilitating the identification and management of a patient's negative emotions or mindsets during a clinical therapy session, comprising the steps of:
(a) engaging the patient in a dialogue to identify the patient's most significant negative emotions or mindsets;
(b) providing a plurality of actual, physical buckets, each to be labeled by the patient or therapist with a name corresponding to one of the identified negative emotions or mindsets;
(c) discussing each labeled bucket between the therapist and the patient, wherein the discussion includes the assignment of tangible objects into each bucket, with each object representing a specific incident, thought, or feeling associated with the corresponding negative emotion or mindset;
(d) tracking the intensity and prevalence of the identified negative emotions or mindsets by analyzing the quantity and nature of objects assigned to each bucket during the course of the therapy session;
(e) utilizing the outcomes of the analysis to guide the therapeutic conversation, aiming to address, mitigate, or understand the underlying causes of the negative emotions or mindsets identified; and
(f) adapting the clinical therapy plan based on the progress observed through the tracking of object assignments to the buckets, to provide a tailored approach to the patient's mental health care.

2. The method of claim 1, wherein the dialogue in step (a) is conducted using a structured interview format that includes a predetermined set of questions designed to elicit information about the patient's negative emotions or mindsets.

3. The method of claim 1, wherein the buckets provided in step (b) are of different colors, each color corresponding to a different negative emotion or mindset.

4. The method of claim 1, wherein the tangible objects in step (c) are selected from a group consisting of: stones, tokens, figures, and written notes.

5. The method of claim 1, wherein the tracking in step (d) includes recording the number of objects in each bucket at predetermined intervals during the therapy session.

6. The method of claim 1, wherein the tracking in step (d) further includes the therapist's observations about the patient's behavior and verbal expressions related to each negative emotion or mindset.

7. The method of claim 1, wherein the outcomes of the analysis in step (e) are documented in a visual chart or graph to facilitate the discussion.

8. The method of claim 1, wherein the adapting of the clinical therapy plan in step (f) includes incorporating specific therapeutic techniques chosen based on the predominant negative emotion or mindset as indicated by the bucket with the most objects.

9. The method of claim 1, further comprising a step of removing objects from a bucket when the patient reports a resolution of the associated negative emotion or mindset, thereby visually representing therapeutic progress.

10. The method of claim 1, wherein the tangible objects are personalized by the patient, providing a means to express their individual experiences related to their negative emotions or mindsets.

11. A method for identifying one or more clinical issues in a patient's mental health, comprising:
a. providing a plurality of actual, physical buckets, wherein each said bucket represents a specific mental health issue selected from the group consisting of: anger, depression, guilt, anxiety, fear, bitterness, and the like;
b. instructing the patient to assign tangible items or tokens into each bucket based on personal experiences, feelings, or thoughts that correspond to the mental health issue represented by said bucket;
c. analyzing the distribution of items or tokens among the buckets by a healthcare professional to identify predominant mental health issues based on the quantity and nature of items or tokens placed in each bucket;
d. engaging in a therapeutic dialogue with the patient focused on the outcomes of the distribution analysis to further explore and understand the patient's mental health issues; and
e. developing a tailored clinical therapy plan based on the identified predominant mental health issues, incorporating strategies for addressing each identified issue as represented by the respective buckets.

12. The method of claim 11, wherein the actual, physical buckets are color-coded, with each color representing a different one of the specific mental health issues.

13. The method of claim 11, wherein the tangible items or tokens are provided by the healthcare professional and are selected for their varied shapes, colors, and textures to facilitate sensory engagement by the patient.

14. The method of claim 11 wherein the instruction to the patient in step b includes a reflective exercise in which the patient writes down the personal experience, feeling, or thought before placing the corresponding item or token in the bucket.

15. The method of claim 11 wherein analyzing the distribution of items or tokens includes counting the number of items or tokens in each bucket and recording the emotional intensity conveyed by each item or token as expressed by the patient.

16. The method of claim 11 wherein the therapeutic dialogue in step d includes the use of cognitive-behavioral therapy techniques to challenge and reframe the thoughts associated with the predominant mental health issues.

17. The method of claim 11 further comprising a step of the healthcare professional providing feedback to the patient about the patterns observed in the distribution of items or tokens among the buckets.

18. The method of claim 11 wherein the tailored clinical therapy plan in step e includes follow-up sessions where the distribution of items or tokens is reviewed and compared to previous sessions to track changes over time.

19. The method of claim 1 further comprising a step f of adjusting the therapy plan in response to a significant change in the distribution of items or tokens indicative of a shift in the patient's mental health issues.

20. The method of claim 1 wherein the buckets are provided with lids, and the step of analyzing the distribution of items or tokens includes a subsequent reveal to the patient, facilitating a moment of insight related to the predominant mental health issues.
